(19) United States
(12) Patent Application Publication
(10) Pub. No.: US 2009/0042718 A1
(43) Pub. Date: Feb. 12, 2009

(54) Direct epoxidation catalyst and process

(76) Inventors: Mark P. Kaminsky, Media, PA (US); Roger A. Grey, West Chester, PA (US); Edrick Morales, West Chester, PA (US)

Correspondence Address: LyondellBasell Industries, 3801 WEST CHESTER PIKE, NEWTOWN SQUARE, PA, 19073, US

(21) Appl. No.: 11/891,460

(22) Filed: Aug. 10, 2007

(51) Int. Cl. B01J 29/068; C07D 301/03

(52) U.S. Cl. 502/74; 502/87; 549/523

(57) ABSTRACT

A catalyst comprising a transition metal zeolite and a noble metal supported on a titania-containing carrier is disclosed. The supported noble metal has a mean mass diameter of from 2 to 200 μm. The catalyst is used in an epoxidation process comprising reacting an olefin, hydrogen, and oxygen. The supported noble metal is well dispersed in the reaction media.

### FIELD OF THE INVENTION

The invention relates to a catalyst comprising a transition metal zeolite and a noble metal supported on a titania-containing carrier. The catalyst is used to produce an epoxide by reacting an olefin, hydrogen, and oxygen.

### BACKGROUND OF THE INVENTION

Direct epoxidation of higher olefins (containing three or more carbons) such as propylene with oxygen and hydrogen has been the focus of recent efforts. For example, the reaction may be performed in the presence of a catalyst comprising gold and a titanium-containing support (see, e.g., U.S. Pat. Nos. 5,623,090, 6,362,349, and 6,646,142), or a catalyst containing palladium and a titanium zeolite (see, e.g., JP 4-352771).

Mixed catalyst systems for olefin epoxidation with hydrogen and oxygen have also been disclosed. For example, Example 13 of JP 4-352771 describes the use of a mixture of titanosilicate and Pd-on-carbon for propylene epoxidation. U.S. Pat. No. 6,008,388 describes a catalyst comprising a noble metal and a titanium or vanadium zeolite, but additionally teaches that the Pd can be incorporated into a support before mixing with the zeolite. The catalyst supports disclosed include silica, alumina, and activated carbon. U.S. Pat. No. 6,498,259 discloses the epoxidation of an olefin with hydrogen and oxygen in a solvent containing a buffer in the presence of a catalyst mixture containing a titanium zeolite and a noble metal catalyst.

In a slurry reaction, it is generally believed to be easier to disperse smaller particles in a liquid than large ones of the same composition. We have found that in order to have well dispersed titania-supported noble metal in a slurry epoxidation process using a mixed catalyst systems, the titania-supported noble metal of large enough particle size needs to be used.

### SUMMARY OF THE INVENTION

The invention is a catalyst comprising a transition metal zeolite and a noble metal supported on a titania-containing carrier. The supported noble metal has a mean mass diameter of from 2 to 200 μm. The catalyst is used in an epoxidation process comprising reacting an olefin, hydrogen, and oxygen.

### DETAILED DESCRIPTION OF THE INVENTION

The invention is a catalyst comprising a transition metal zeolite. Zeolites are porous crystalline solids with well-defined structures. Generally they contain one or more of Si, Ge, Al, B, P, or the like, in addition to oxygen. Many zeolites occur naturally as minerals and are extensively mined in many parts of the world. Others are synthetic and are made commercially for specific uses. Zeolites have the ability to act as catalysts for chemical reactions which take place mostly within their internal cavities. Transition metal zeolites are zeolites comprising transition metals in framework. A transition metal is a Group 3-12 element. The first row of transition metals are from Sc to Zn. Preferred transition metals are Ti, V, Mn, Fe, Co, Cr, Zr, Nb, Mo, and W. More preferred are Ti, V, Mo, and W. Most preferred is Ti.

Preferred titanium zeolites are titanium silicates (titanosilicates). Preferably, they contain no element other than titanium, silicon, and oxygen in the lattice framework (see R. Szostak, “Non-aluminosilicate Molecular Sieves,” in Molecular Sieves: Principles of Synthesis and Identification (1989), Van Nostrand Reinhold, pp. 205-82). Small amounts of impurities, e.g., boron, iron, aluminum, phosphorous, copper, and the like, and mixtures thereof, may be present in the lattice. The amount of impurities is preferably less than 0.5 wt. %, more preferably less than 0.1 wt. %. Preferred titanium silicates will generally have a composition corresponding to the following empirical formula: xTiO₂.(1−x)SiO₂, where x is between 0.0001 and 0.5000. More preferably, the value of x is from 0.01 to 0.125. The molar ratio of Si to Ti in the lattice framework of the zeolite is advantageously from 9.5:1 to 99:1, most preferably from 9.5:1 to 60:1. Particularly preferred titanium zeolites are titanium silicalites (see Catal. Rev.-Sci. Eng., 39(3) (1997) 209). Examples of these include TS-1 (titanium silicalite-1, a titanium silicalite having an MFI topology analogous to that of the ZSM-5 aluminosilicate), TS-2 (having an MEL topology analogous to that of the ZSM-11 aluminosilicate), and TS-3 (as described in Belgian Pat. No. 1,001,038). Titanium zeolites having framework structures isomorphous to zeolite beta, mordenite, ZSM-12, MCM-22, MCM-41, and MCM-48 are also suitable for use. Examples of MCM-22, MCM-41, and MCM-48 zeolites are described in U.S. Pat. Nos. 4,954,325, 6,077,498, and 6,114,551; Maschmeyer, T., et al, Nature 378(9) (1995) 159; Tanev, P. T., et al., Nature 368 (1994) 321; Corma, A., J. Chem. Soc., Chem. Commun. (1998) 579; Wei D., et al., Catal. Today 51 (1999) 501). The most preferred is TS-1.

Suitable transition metal zeolites include transition metal zeolite crystals and other formed transition metal zeolite particles. Formed transition metal zeolite particles may be prepared by many standard techniques known to a person skilled in the art. Spray drying is a preferred forming technique. Spray drying is a suspended particle processing system that utilizes liquid atomization to create droplets which are dried to individual particles while moving in a gaseous medium (see K. Maters, Spray Drying In Practice, SparyDryConsultant International ApS (2002) pp. 1-15). Spray drying is known in forming zeolites, including titanium zeolites (see, e.g., U.S. Pat. Nos. 4,954,653, 4,701,428, 5,500,199, 6,524,984, and 6,106,803).

The transition metal zeolite (crystals or formed particles) preferably has a mean mass diameter of from 2 to 200 μm, more preferably from 10 to 100 μm, most preferably from 15 to 50 μm. Mean mass diameter is the arithmetic mean diameter of all the particle masses forming the entire population (R. Trottier and S. Wood, “Particle Size Measurement,” in Kirk-Othmer Encyclopedia of Chemical Technology, online edition, 2007).

A transition metal zeolite containing templating agent may be formed to particles. A transition metal zeolite is generally prepared in the presence of an organic templating agent (see, e.g., U.S. Pat. No. 6,849,570). Suitable templating agents include alkyl amines, quaternary ammonium compounds, etc. When a zeolite is crystallized, it usually contains organic templating agent within its pores than can be removed by calcination or solvent extraction. Zeolites, with or without containing templating agents, may be spray dried.

A binder is preferably used in spray drying the transition metal zeolite. A binder helps to improve the mechanical strength and/or the physical properties of the spray-dried particles (e.g., crushing strength, surface area, pore size, pore volume). Sometimes the binder can modify the chemical properties (e.g., acidity, basicity) of the transition metal zeolite and its catalytic activity.

Suitable binders include metal oxides, non-metal oxides, mixed oxides, clays, and the like. Examples of suitable binders include silicas, aluminas, titanias, magnesias, silica-aluminas, silica-titanias, montmorillonites, kaolins, bentonites, halloysites, dickites, nacrites, and anauxites, and the like, and mixtures thereof. Examples of clays can be found in “Chapter 2. Clay as Potential Catalyst Material,” Zeolite, Clay, and Heteropoly Acid in Organic Reactions (1992) Kodansha Ltd., Tokyo. Preferred binders are silicas, aluminas, titanias, silica-aluminas; silica-titanias, kaolins, and mixtures thereof. More preferred are silicas, aluminas, titanias, and mixtures thereof.

Precursors of binders are often used in preparing the mixture for spray drying. For example, silica may be introduced into the mixture as a silica sol (Healy, T. W., “Stability of Aqueous Silica Sols,” in The Colloid Chemistry of Silica (1994) American Chemical Society). Similarly, other binder precursors such as orthosilicic esters, alkoxysilanes, alkoxytitanates, alkoxyaluminates can also be used. Specific examples are tetramethoxysilane, tetraethoxysilane, tetrapropoxysilane, tetrabutoxysilane, and analogous tetraalkoxytitanium, and trialkoxyaluminium compounds. Precursors are converted to the corresponding binder during mixing, spray drying, or calcination.

The catalyst comprises a noble metal. Suitable noble metals include gold, silver, platinum, palladium, iridium, ruthenium, osmium, rhenium, rhodium, and mixtures thereof. Preferred noble metals are Pd, Pt, Au, Re, Ag, and mixtures thereof. Palladium, gold, and their mixtures are particularly desirable. Typically, the amount of noble metal present in the catalyst will be in the range of from 0.01 to 20 wt. %, preferably 0.1 to 5 wt. %.

There are no particular restrictions regarding the choice of the noble metal compound or complex used as the source of the noble metal. Suitable compounds include nitrates, sulfates, halides (e.g., chlorides, bromides), carboxylates (e.g., acetate), and amine or phosphine complexes of noble metals (e.g., palladium(II) tetraammine bromide, tetrakis(triphenylphosphine) palladium(0)).

The weight ratio of the transition metal zeolite to noble metal is not particularly critical. However, a transition metal zeolite to noble metal weight ratio of from 10:1 to 10,000:1 (grams of transition metal zeolite per gram of noble metal) is preferred.

The noble metal is supported on a titania-containing carrier. The combination of the noble metal and the titania-containing carrier is referred to as “supported noble metal.” The carrier contains, preferably at least 80 wt. % titania, more preferably at least 90 wt. % titania.

There are many suitable methods for supporting the noble metal on the carrier, including, e.g., impregnation, adsorption, ion-exchange, and precipitation.

The supported noble metal has a mean mass diameter of from 2 to 200 μm. Preferably, its mean mass diameter is from 5 to 150 μm. More preferably, it is from 10 to 100 μm. Most preferably, it is from 15 to 50 μm. A supported noble metal of such particle sizes can be well dispersed in a reaction mixture and it is suitable for a slurry process.

Preferably, the transition metal zeolite particles and the supported noble metal are similar in particle sizes. Particularly preferred catalysts comprise transition metal zeolite particles and the supported noble metal each having a mean mass diameter of 15 to 50 μm.

The carrier may be prepared from a titania source. Suitable titania sources include titania powder, titania sol, and other titanium compounds (e.g., titanium halides, titanium alkoxides, chelated titanium complexes). Spray drying is a preferred technique to prepare titania-containing carriers.

The supported noble metal may contain other components such as silica, alumina, silica-alumina, clays, alkali metals, alkaline earth metals, lead, phosphates, halide, nitrate, sulfate, and the like. Such components may help to improve the mechanical, physical, and chemical properties of the supported noble metal. These components may be incorporated into the carrier during its preparation. Alternatively, they may be added on the carrier after the carrier is formed. For example, they may be added before, after, or during the addition of the noble metal with standard techniques including impregnation, co-precipitation, adsorption, and ion-exchange.

The invention also includes an epoxidation process comprising reacting an olefin, hydrogen, and oxygen in the presence of the catalyst of the invention.

An olefin is used in the process. Suitable olefins include any olefin having at least one carbon-carbon double bond, and generally from 2 to 60 carbon atoms. Preferably the olefin is an acyclic alkene of from 2 to 30 carbon atoms; the process is particularly suitable for epoxidizing C₂-C₆ olefins. More than one double bond may be present in the olefin molecule, as in a diene or triene. The olefin may be a hydrocarbon or may contain functional groups such as halogen, carboxyl, hydroxy, ether, carbonyl, cyano, or nitro groups, or the like. In a particularly preferred process, the olefin is propylene and the epoxide is propylene oxide.

Oxygen and hydrogen are required. Although any sources of oxygen and hydrogen are suitable, molecular oxygen and molecular hydrogen are preferred. The molar ratio of hydrogen to oxygen can usually be varied in the range of H₂:O₂=1:100 to 5:1 and is especially favorable at 1:5 to 2:1. The molar ratio of oxygen to olefin is usually 1:1 to 1:20, and preferably 1:1.5 to 1:10. Relatively high oxygen to olefin molar ratios (e.g., 1:1 to 1:3) may be advantageous for certain olefins.

In addition to the olefin, oxygen, and hydrogen, an inert gas is preferably used in the process. Any desired inert gas can be used. Suitable inert gases include nitrogen, helium, argon, and carbon dioxide. Saturated hydrocarbons with 1-8, especially 1-6, and preferably 1-4 carbon atoms, e.g., methane, ethane, propane, and n-butane, are also suitable. Nitrogen and saturated C₁-C₄ hydrocarbons are preferred inert gases. Mixtures of inert gases can also be used. The molar ratio of olefin to gas is usually in the range of 100:1 to 1:10 and especially 20:1 to 1:10.

The process may be performed in a continuous flow, semi-batch, or batch mode. A continuous flow process is preferred.

It is advantageous to work at a pressure of 1-200 bars. The process is carried out at a temperature effective to achieve the desired olefin epoxidation, preferably at temperatures in the range of 0-200° C., more preferably, 20-150° C. Preferably, at least a portion of the reaction mixture is a liquid under the reaction conditions.

A reaction solvent is preferably used in the process. Suitable reaction solvents are liquid under the reaction conditions. They include, for example, oxygen-containing hydrocarbons such as alcohols, aromatic and aliphatic solvents such as toluene and hexane, nitriles such as acetonitrile, carbon dioxide, and water. Suitable oxygenated solvents include alcohols, ethers, esters, ketones, carbon dioxide, water, and the like, and mixtures thereof. Preferred oxygenated solvents include water and lower aliphatic C₁-C₄ alcohols such as methanol, ethanol, isopropanol, tert-butanol, and mixtures thereof.

Where a reaction solvent is used, it may be advantageous to use a buffer. The buffer is employed in the reaction to inhibit the formation of glycols or glycol ethers during the epoxidation, and it can improve the reaction rate and selectivities. The buffer is typically added to the solvent to form a buffer solution, or the solvent and the buffer are added separately. Useful buffers include any suitable salts of oxyacids, the nature and proportions of which in the mixture are such that the pH of their solutions preferably ranges from 3 to 12, more preferably from 4 to 10, and most preferably from 5 to 9. Suitable salts of oxyacids contain an anion and a cation. The anion may include phosphate, carbonate, bicarbonate, sulfate, carboxylates (e.g., acetate), borate, hydroxide, silicate, aluminosilicate, or the like. The cation may include ammonium, alkylammonium (e.g., tetraalkylammoniums, pyridiniums), alkylphosphonium, alkali metal, and alkaline earth metal ions, or the like. Examples include NH₄, NBu₄, NMe₄, Li, Na, K, Cs, Mg, and Ca cations. The preferred buffer comprises an anion selected from the group consisting of phosphate, carbonate, bicarbonate, sulfate, hydroxide, and acetate; and a cation selected from the group consisting of ammonium, alkylammonium, alkylphosphonium, alkali metal, and alkaline earth metal ions. Buffers may preferably contain a combination of more than one suitable salt. Typically, the concentration of the buffer in the solvent is from 0.0001 M to 1 M, preferably from 0.0005 M to 0.3 M. The buffer may include ammonium hydroxide which can be formed by adding ammonia gas to the reaction system. For instance, one may use a pH=12-14 solution of ammonium hydroxide to balance the pH of the reaction system. More preferred buffers include alkali metal phosphates, ammonium phosphate, and ammonium hydroxide. The ammonium phosphate buffer is particularly preferred.

Following examples illustrate the invention.

### EXAMPLE 1

### Pd/Au/Titania (Catalyst A)

An aqueous slurry is prepared with TiO₂ (Millennium Inorganic Chemicals S5-300B). The slurry contains 17.5 wt. % titania. The slurry is spray dried with a Mobile. Minor Spray Dryer (Niro Inc.) configured for a two-point powder discharge and a rotary atomizer. The drying chamber has an inside diameter of 2.7 feet and a 2-feet cylindrical height and a 60-degree angle conical bottom. A Watson Marlow peristaltic pump (model 521CC) is used to feed the slurry to the atomizer wheel and control the exit temperature. The main product is collected at the bottom port of the drying chamber and fines are routed to the cyclone collector. Air is used as drying/process gas at a flow rate of 80 kg/h. The inlet temperature is set at 220° C. The atomizer wheel is set at 27,000 RPM. A Watson Marlow peristaltic pump is used to evaporate de-ionized water and control the exit temperature of the drying chamber to 95° C. The product is collected at the bottom of the drying chamber. Its mean mass diameter is 24 μm. The spray-dried titania is calcined in air at 700° C. The calcined spray-dried titania has a surface area of 40 m²/g.

A round-bottom flask is charged with 25 mL of deionized water. To the water, 0.265 g of aqueous sodium tetrachloro aurate (20.74 wt. % gold), 0.275 g of disodium palladium tetrachloride, and 10 g of calcined spray-dried titania prepared above is added. To this slurry, 0.26 g of solid sodium bicarbonate is added. The slurry is agitated by rotating the flask at 25 rpm at a 45-degree angle for 4 h at 40° C., then filtered. The solids are washed once with 25 mL of deionized water. The solids are then calcined in air by heating at 10° C./min to 110° C. and holding at 110° C. for 4 h, then heating at 2° C./min to 300° C. and holding at 300° C. for 4 h. The calcined solids are washed with deionized water (25 mL×8). The solids are calcined in air by heating at 10° C./min to 110° C. for 4 h, and then at 2° C./min to 550° C. and holding at 550° C. for 4 h. The solids are then transferred to a quartz tube and treated with a hydrogen/nitrogen (mole ratio 4:96, 100 mL/h) gas at 100° C. for 1 h, followed by nitrogen for 30 min as the catalyst cools from 100° C. to 30° C. The solids obtained (Catalyst A) contain 0.95 wt. % palladium, 0.6 wt. % gold, 58 wt. % titanium, and less than 20 ppm chloride.

### EXAMPLE 2

### Propylene Epoxidation with Spray-Dried TS-1 and Catalyst A

Titanium silicalite-1 (TS-1, mean mass diameter 0.2 μm) crystals are prepared by following procedures disclosed in U.S. Pat. Nos. 4,410,501 and 4,833,260, and calcined in air at 550° C. Spray-dried TS-1 is prepared by following procedures disclosed in U.S. Pat. No. 5,500,199. It is calcined in air at 550° C. The calcined spray-dried TS-1 contains approximately 80 wt. % TS-1 and 20 wt. % silica.

A 300-mL stainless steel reactor is charged with Catalyst A (0.725 g) and spray-dried TS-1 (10 g). A hollow, DispersiMax agitator shaft and impeller (Rushton turbine) runs down from the head of the reactor and is turned with a Magnadrive coupling. The agitator typically runs at 500 to 1200 rpm to ensure proper back-mixing of gas and liquid with the catalyst. The reactor is charged with methanol/water (80/20 weight ratio, 199 mL) containing 0.010 M ammonium dihydrogen phosphate (referred to as solvent/buffer solution). A gas feed containing 4.5 mol. % oxygen, 2.25 mol. % hydrogen, 86.4 mol. % nitrogen, 6.36 mol. % propylene, and 0.45 mol. % methane is flowed through the reactor (flow rate 6670 mL/min). Electronic mass flow controllers are used for the gases and a HPLC piston type pump is used to pump the solvent/buffer solution. Dip tubes direct the gas and liquid feed to near the bottom of the reactor where they pass through metal fritted filters to break up gas bubbles upon entering the reactor. The reactor pressure is 500 psig. The product gas and liquid exit the reactor through fritted metal filters (0.5 micron) in the vent lines. The filters are 2 inches below the reactor head. The filters control the liquid level in the reactor by having the liquid and vapor drain through them. The product mixture then goes to a gas/liquid separator. The gas is fed to an on-line gas chromatograph (GC) for analysis and the liquid is injected to both an on-line and an off-line GC. The products formed include propylene oxide (PO), propane, and derivatives of propylene oxide such as propylene glycol, propylene glycol monomethyl ethers, dipropylene glycol, and dipropylene glycol methyl ethers. The reaction proceeds smoothly for 1400 h. The average catalyst productivity is 0.25 g POE per gram catalyst per hour. The catalyst productivity is defined as the grams of propylene oxide (PO) formed (including PO that is subsequently reacted to form PO derivatives) per gram of catalyst per hour. PO and PO equivalent, POE (mole)=moles of PO+moles of PO units in the PO derivatives. PO/POE selectivity=(moles of PO)/(moles of POE)×100. Propylene to POE selectivity=(moles of POE)/(moles of propane formed+moles of POE)×100.

After 1400 h on stream, the used catalyst admixture appears homogeneously mixed. There is no sign of clumping or segregation of particles. Elemental analyses of the mixture is consistent with that it is a uniform mixture of Catalyst A and spray-dried TS-1.

### COMPARATIVE EXAMPLE 3

### Pd/Au/Titania (Catalyst B)

A round bottom flask is charged with 40 mL of deionized water, 0.4 g of aqueous sodium tetrachloro aurate (20.74 wt. % gold), 0.41 g of disodium palladium tetrachloride, and 15 g of powdered titania (mean mass diameter 1.2 μm, air calcined at 700° C., surface area 30 m²/g). To the slurry, 0.4 g of solid sodium bicarbonate is added. The slurry is agitated by rotation of the flask at 25 rpm at a 45 degree angle for 4 h at 40° C., then filtered. The solids are washed once with 40 mL of deionized water, then calcined in air by heating at 10° C./min to 110° C. and holding at 110° C. for 4 h, then heating at 2° C./min to 300° C. and holding at 300° C. for 4 h. The calcined solids are then washed with more deionized water (40 mL×8). The solids are calcined in air by heating at 10° C./min to 110° C. for 4 h and holding at 110° C. for 4 h, then heating at 2° C./min to 550° C. and holding at 550° C. for 4 h. The solids are transferred to a quartz tube and treated with a hydrogen/nitrogen (mole ratio 4:96, 100 mL/h) gas at 100° C. for 1 h, then with flowing nitrogen for 30 min as the catalyst cools from 100° C. to 30° C. The final solids (Catalyst B) contains 0.88 wt. % palladium, 0.6 wt. % gold, 58 wt. % titanium, and less than 20 ppm chloride.

### COMPARATIVE EXAMPLE 4

### Propylene Epoxidation with Spray-Dried TS-1 and Catalyst B

The procedure of Example 2 is repeated except that Catalysts B is used instead of Catalyst A. The average catalyst productivity is 0.17 g POE per gram catalyst per hour. Several back-washes of the filter are performed. POE productivity increases significantly after these back-washes, but declines quickly. The test is terminated after 400 h. The catalyst in the reactor is a mixture of black clumps and a white powder. The black clumps are aggregates of Catalyst B and the white powder is spray-dried TS-1.

This test shows that Catalyst B tends to aggregate in the reaction media, thus the dispersion of the Catalyst B can not be maintained due to its small particle size. The lower productivity obtained than that in Example 2 is most probably due to the poor dispersion of Catalyst B in the reaction media.

We claim:

1. A catalyst comprising a transition metal zeolite, and a supported noble metal having a mean mass diameter of from 2 to 200 μm, wherein the supported noble metal comprises a noble metal and a titania-containing carrier.

2. The catalyst of claim 1 wherein the supported noble metal has a mean mass diameter of from 5 to 150 μm.

3. The catalyst of claim 1 wherein the supported noble metal has a mean mass diameter of from 10 to 100 μm.

4. The catalyst of claim 1 wherein the supported noble metal has a mean mass diameter of from 15 to 50 μm.

5. The catalyst of claim 1 wherein the transition metal zeolite is a titanium zeolite.

6. The catalyst of claim 1 wherein the transition metal zeolite is TS-1.

7. The catalyst of claim 1 wherein the noble metal is selected from the group consisting of gold, silver, platinum, palladium, iridium, ruthenium, rhenium, rhodium, osmium, and mixtures thereof.

8. The catalyst of claim 1 wherein the noble metal is palladium, gold, or a palladium-gold mixture.

9. The catalyst of claim 1 wherein the carrier contains at least 80 wt. % titania.

10. The catalyst of claim 1 wherein the carrier contains at least 90 wt. % titania.

11. An epoxidation process comprising reacting an olefin, hydrogen, and oxygen in the presence of the catalyst of claim 1.

12. The process of claim 11 wherein the supported noble metal has a mean mass diameter of from 10 to 100 μm.

13. The process of claim 11 wherein the supported noble metal has a mean mass diameter of from 15 to 50 μm.

14. The process of claim 11 wherein the transition metal zeolite has a mean mass diameter of from 15 to 50 μm.

15. The process of claim 11 wherein the transition metal zeolite is a titanium zeolite.

16. The process of claim 11 wherein the transition metal zeolite is TS-1.

17. The process of claim 11 wherein the noble metal is selected from the group consisting of gold, silver, platinum, palladium, iridium, ruthenium, rhenium, rhodium, osmium, and mixtures thereof.

18. The process of claim 11 wherein the noble metal is palladium, gold, or a palladium-gold mixture.

19. The process of claim 11 wherein the carrier contains at least 90 wt. % titania.

20. The process of claim 11 wherein the olefin is propylene.
